(19) United States
(12) Patent Application Publication
(10) Pub. No.: US 2010/0168050 A1
(43) Pub. Date: Jul. 1, 2010

(54) COMPOUNDS HAVING ANTIANGIOGENIC ACTIVITY

(75) Inventor: Maria Elena FERRERO, Milano (IT)

Correspondence Address: YOUNG & THOMPSON, 209 Madison Street, Suite 500, Alexandria, VA, 22314, US

(73) Assignee: MEDESTEA INTERNAZIONALE S.P.A, Torino (IT)

(21) Appl. No.: 12/626,356

(22) Filed: Nov. 25, 2009

Related U.S. Application Data
(63) Continuation-in-part of application No. 10/589,621, filed on Oct. 6, 2006, now abandoned.

(30) Foreign Application Priority Data
Feb. 17, 2004 (IT) MI2004A000255

(51) Int. Cl. A61K 31/7076; A61P 35/00; A61P 35/02

(52) U.S. Cl. 514/47

(57) ABSTRACT

The use of o-ATP as a pharmacological agent useful for the treatment of diseases in whose onset or progression angiogenesis is involved.

### CROSS REFERENCE TO RELATED APPLICATIONS

This application is a continuation-in-part of U.S. application Ser. No. 10/589,621 filed on Aug. 16, 2006, which is the 35 U.S.C. 371 National Stage of International Application PCT/EP03/04720 filed on May 6, 2003, which claimed priority to Italian Application MI2002A000960 filed May 7, 2002. The entire contents of each of the above-identified applications are hereby incorporated by reference.

### FIELD OF THE INVENTION

The present invention relates in general to substances that act on angiogenesis. More precisely, the invention relates to the use of o-ATP for the treatment of pathologies that require inhibition of angiogenesis.

### BACKGROUND OF THE INVENTION

Angiogenesis

Proliferation of endothelial cells is responsible for the process of formation of new blood vessels, known as angiogenesis. The newly formed vessels provide nutrients and oxygen to the cells of the tissue wherein angiogenesis occurs. The angiogenetic process is useful, for example, for wound repair, since regenerating tissues necessitate a proper blood supply. On the contrary, angiogenesis is detrimental in the case of tumor diseases, because blood supply facilitates the proliferation of tumor cells. In addition, neoangiogenesis is detrimental when develops into the atherosclerotic plaques; in fact in these structures the generation of new vessels due to VEGF (vascular endothelial growth factor) production by endothelial and other cells, as monocytes/macrophages, supports the preservation of the same plaques. Therefore, the inhibition of endothelial cells proliferation, or anti-angiogenic activity, is of remarkable interest in antitumor and antiatherosclerotic therapies.

o-ATP's Biological Activity

The oxidized form of ATP, known as o-ATP, is characterized by the presence of two aldehyde groups at the positions 2′ and 3′ of the ribofuranosyl ring. It can be prepared by treatment of ATP with a periodic acid salt, as disclosed by P. N. Lowe et al., “Preparation and chemical properties of periodate-oxidized adenosine triphosphate and some related compounds”, Biochemical Society Transactions, vol. 7:1131-1133, 1979.

o-ATP is commonly used as an affinity marker for nucleotide enzymatic sites (Easterbrook-Smith, B., Wallace, J. C. & Keech, D. B. (1976) Eur. J. Biochem. 62, 125-130), thanks to its ability to react with non-protonated lysine residues that are present in the nucleotide sites, forming Schiff's bases or dihydromorpholine-derivatives (Colman, R. F. (1990) in The Enzymes—Sigman, D. S., and Boyer, P. D., eds—Vol 19, pp. 283-323, Academic Press, San Diego). It has also been used to study platelet activation and inhibit ATP-induced stimulation of chicken muscle (Pearce, P. H., Wright, J. M. Egan. C. M. & Scrutton, M. C. (1978) Eur. J. Biochem. 88, 543-554; Thomas, S. A., Zawisa, M. J., Lin, X. & Hume, R. I. (1991) Br. J. Pharmacol. 103, 1963-1969). Furthermore, in macrophage cell lines, o-ATP proved able to block ATP-induced permeabilization of the plasma membrane, reduce the hydrolysis level of exogenous ATP by membrane ecto-ATPases, and inhibit ATP-induced cell swelling, vacuolization and lysis (Murgia et al. The Journal of Biological Chemistry, (1993) by The American Society for Biochemistry and Molecular Biology, inc., Vol. 268, No. 11, pp 8199). It has been suggested that o-ATP has an antagonist activity on the purinergic receptor P2z/P2X7, due to the fact that IL-1β (interleukin 1β) release (which is dependent on LPS=lipopolysaccharide) from microglia cells expressing P22/P2X7 is selectively inhibited by o-ATP (Ferrari D. et al., J. Exp. Med., (1997) Vol. 185, N. 3, Pag. 579-582).

### PRIOR ART

WO 02/11737, in the name of the Applicant, discloses o-ATP anti-inflammatory and analgesic effect, using unilateral inflammation of rat paw caused by intraplantar injection of complete Freund's adjuvant (CFA) as the experimental model.

### BRIEF SUMMARY OF THE INVENTION

In vitro assays on human umbilical vein endothelial cells (HUVEC) showed that o-ATP induces a significant reduction of their proliferative capacity, even in the presence of a mitogen. The effect of o-ATP resulted higher than that induced by vasostatin, a known anti-angiogenic compound.

The antiangiogenic effects of oATP were also demonstrated in experiments where either non-stimulated or TNFα-stimulated PBMC transendothelial migration was inhibited by the addition of oATP. Furthermore, oATP was shown to inhibit cell-proliferation, by inducing apoptosis in RMA (lymphoma) cells in culture, and proved effective in an animal model of lymphoma, significantly reducing tumor growth in mice carrying a T-cell lymphoma.

In separate experiments, o-ATP and two P2X7 receptor antagonists (the pyridoxal phosphate-6-azophenil-2′,4′-disulphonic acid (PPADS), a non-specific P2X7 antagonist, and 1-(N,O-bis[5-isoquinolinesulphonyl]-N-methyl-L-tyrosyl)-4-phenylpiperazine (KN62), a potent P2X7 antagonist, especially at the human receptor) were tested for their ability to inhibit the proliferation of human umbilical vein endothelial cells (HUVEC) and to induce apoptosis in a promyelocytic leukemia cell line (HL60). Furthermore o-ATP was assayed for its ability to modulate the expression of TNFalpha receptors TNFR1 and TNFR2. The results suggest that:
1) compared to the reference P2X7 antagonists, oATP-induced inhibition of endothelial cell proliferation is significantly higher; neither oATP nor the P2X7 antagonists induce apoptosis in the same cells;
2) oATP downregulates the expression of TNFalpha R1;
3) compared to the P2X7 antagonists, oATP-induced apoptosis of HL60 cells is significantly higher.

The effects described under 1) and 2) are predictive of a higher efficacy of oATP to counteract the angiogenic process, which involves the proliferation of endothelial cells and an inflammatory state caused by TNFalpha, whereas the effects described under 3) are indicative of a higher anti-leukemia effectiveness of oATP compared to P2X7 antagonists.

These results suggest the specificity of the oATP antitumor effect compared to a reference compound that, like oATP, is known to interact with P2X7 nucleotide receptor but, unlike oATP, is unable or much less capable to inhibit endothelial cell proliferation and to induce apoptosis in human promyelocytic leukemia HL60 cells.

It is therefore object of the present invention a method for treating angiogenesis-related diseases in a subject in need thereof, wherein said method comprises administering to said subject an effective amount of adenosine-5′-triphosphate-2′,3′-dialdehyde (oATP).

### BRIEF DESCRIPTION OF THE DRAWINGS

FIG. 1 shows the results of a proliferation assay.

FIG. 2 shows the results of a permeability assay.

FIG. 3 shows the reduction in cell growth number by 500 microM oATP treatment.

FIG. 4 shows the transendothelial migration assay.

FIG. 5 shows the sensitivity of RMA wild type cell line to different concentrations of oATP evaluated as percentage of AnnexinV-PI positive cells at 24 and 48 hours of treatment.

FIG. 6 shows the sensitivity of lymphocytes from buffy-coat (PMBC), activated or not with PHA, at different concentrations of oATP, evaluated as percentage of AnnexinV-PI positive cells at 24 hours of treatment.

FIG. 7 shows tumor growth after 20 days from RMA cell injection at different levels of oATP treatments.

FIG. 8 compares the effects of o-ATP treatment on cell growth number to a control (NT) and treatment by ATP, PPADS, a non-specific P2X7 antagonist, and KN62, a potent P2X7 antagonist.

FIG. 9 compares apoptosis induction on human umbilical vein endothelial cells (HUVEC) by the different treatments NT, ATP, o-ATP, PPADS and KN62.

FIG. 9a illustrates the effect of o-ATP and human TNFalpha on the expression of the TNFalpha receptor 1 (TNFR1) on HUVEC.

FIG. 10 compares cell apoptosis induction on HL60 in the control (NT) and the different treatments with ATP, o-ATP, PPADS, KN62, ATP+o-ATP, ATP+PPADS, and ATP+KN62.

FIG. 11 compares the effects of the control (NT) and the different treatments with ATP, o-ATP, PPADS, KN62, ATP+o-ATP, ATP+PPADS, and ATP+KN62 on the apoptosis/necrosis induction in the HL60 cells.

### DETAILED DESCRIPTION OF THE INVENTION

Angiogenesis-related diseases are those involving angiogenesis in their onset or progression and include neovascularization-induced ocular diseases, such as diabetic retinopathy, macular degeneration, proliferative vitreoretinopathy, glaucoma, atherosclerotic processes and tumors, such as carcinomas, lymphomas, leukemia, sarcomas, melanomas, gliomas, neuroblastomas and other solid tumors.

In a particularly preferred embodiment, the invention provides a method for treating a tumor disease selected from lymphoma and leukemia in a subject in need thereof, comprising administering to said subject an effective amount of adenosine-5′-triphosphate-2′,3′-dialdehyde.

For therapeutical use, o-ATP can be formulated with pharmaceutically acceptable carriers and excipients, and administered through the oral, topical or parenteral route. Pharmaceutical forms suitable for the different administration routes comprise tablets, pills, capsules, granulates, powders, suppositories, syrups, solutions, suspensions, creams, ointments, gels, pastes, lotions, emulsions, sprays. Pharmaceutical compositions can be prepared as described in Remington's Pharmaceutical Sciences Handbook, Mack Pub. Co., NY, USA, XVII Ed. The amount of active substance per dose unit ranges from 0.01 to 100 mg per Kg of body weight, to be administered once a day or more according to the type and severity of the pathology. In general the daily dose will range from 1 to 300 mg, preferably from 10 to 100 mg.

In another embodiment, the invention refers to combined preparations of o-ATP and other biologically active substances for the treatment of angiogenesis-mediated pathologies. According to a preferred embodiment, o-ATP is used in combination with antitumor substances such as alkaloids, antibiotics, cytotoxic or cytostatic compounds, antimetabolites, antihormonal agents, alkylating agents, peptides, biological response modulators, cytokines. Alternatively, oATP is used in combination with antiatherosclerotic substances, preferably with lipid lowering drugs or statins.

The different active substances can be administered either simultaneously or separately. The choice of the specific combination of active substances, their dosage and way of administration depend on the specific disease, its resistance to pharmacological treatments, patient's tolerance and other variables to be determined on a case by case basis.

### Example 1

### Proliferation Assay

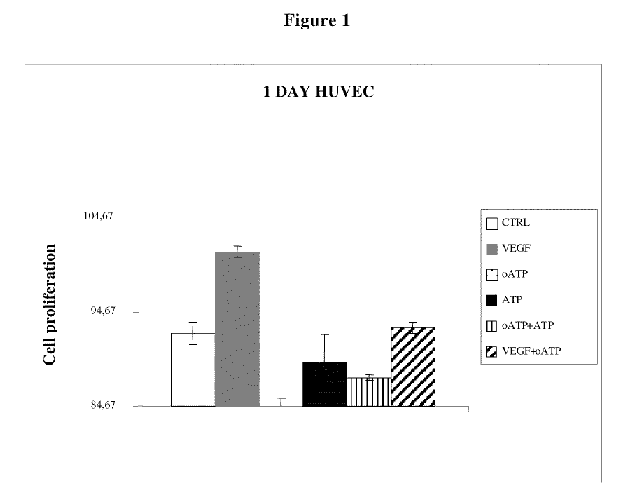

Human endothelial Cells (HUVEC) were isolated from umbilical vein, counted and seeded in a constant number in a 96-well plate. The cells were cultured as described (Jaffe, E. A. (1984) Biology of Endothelial Cells, Martinus Nighoff Publisher, Boston, USA, pp. 1-260), with or without (control) VEGF (50 ng/ml), in the presence of o-ATP (100 μM), and o-ATP+VEGF. After 24 hours cultivation with or without stimulus, the cells were washed and counted with an optical microscope using a Burker chamber. The results are reported in FIG. 1 and represent the mean±SD of 10 experiments.

### Example 2

### Permeability Assay

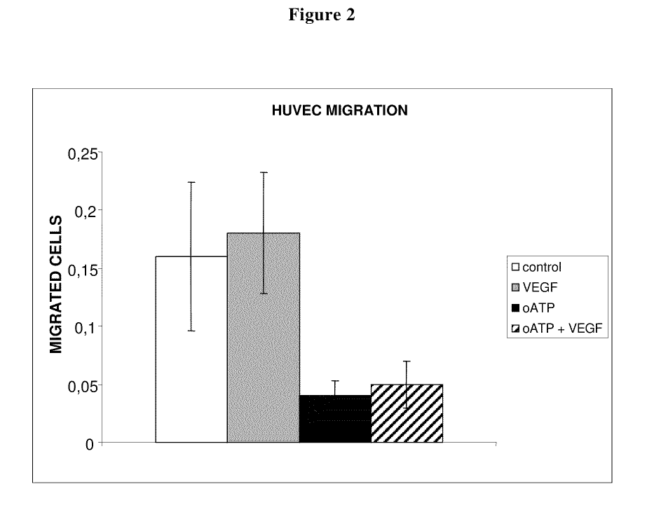

Transwell chambers for cell cultures (polycarbonate filters 0.4 μm, Costar) were used. In short, confluent endothelial cells, in monolayer, were exposed to VEGF, o-ATP, ATP (300 μM), ATP+o-ATP, o-ATP+VEGF (at the previously indicated concentrations) for 1 hr and thoroughly washed. Albumin marked with ¹²⁵I (NEN, Boston, Mass.) was added to the upper compartment; cold albumin (1.5 mg/ml) was added to the culture medium to minimize transcytosis. One hour after the addition of ¹²⁵I-labelled albumin to each well, samples were taken from the lower compartment. The radioactivity of the samples was measured with a gamma counter (Packard, Sterling, Va.). The results, reported in FIG. 2, represent the mean±SD of 10 independent experiments and are expressed as percentage of migrated endothelial cells.

### Example 3

HUVEC were isolated from human umbilical cord by collagenase treatment and cultured in 1% gelatine-coated flasks using endotoxin-free Medium 199, containing 20% heat-inactivated fetal bovine serum, 1% bovine retinal-derived growth factor, 90 microg/ml heparin, 100 IU/ml penicillin and 100 microg/ml streptomycin. All experiments were carried out with HUVEc at the passage 1-4.

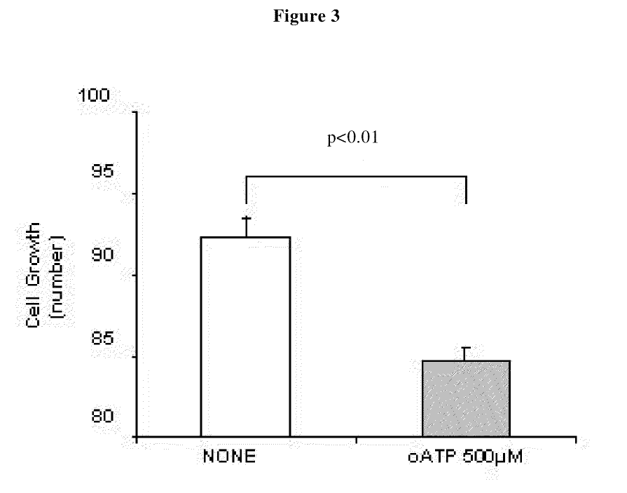

We used oATP at the concentration of 500 microM. HUVEC were treated with oATP over night, washed and fixed with glutaraldeide 2% in PBS. The cells were colored with crystal violet 0.1%, washed and dried. The dye solubilization was performed with acetic acid 10% and the absorbance was measured spectrophotometrically at 595 nm, using a microplate reader. The optical density was proportional to the number of cells. As reported in the FIG. 3, cell growing number is significantly reduced by 500 microM oATP treatment (mean±SEM of 7 experiments).

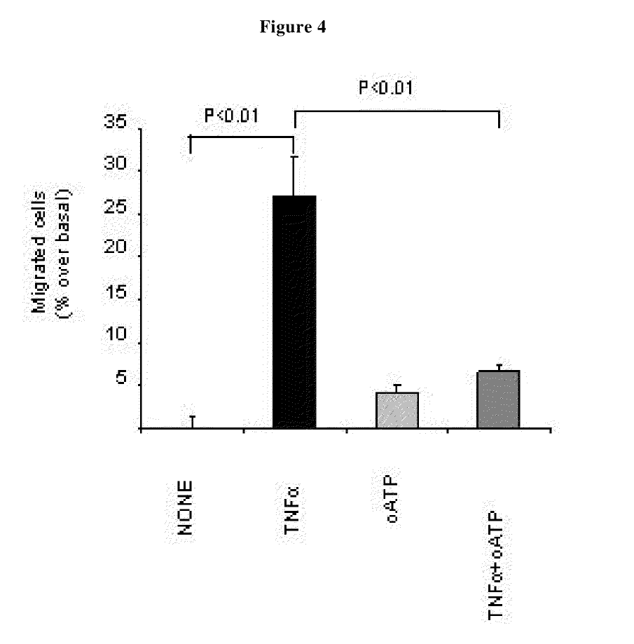

VEGF is a prototypic angiogenetic factor which induces endothelial cell proliferation, angiogenesis and capillary permeability. The latter is evidenced by the assay of transendothelial migration. It is known that VEGF increases transendothelial migration. Migration assay was performed using a Transwell double chamber system (5 micrometer polycarbonate membrane). HUVEC (5×10⁴ cells/well) were seeded on the filter, in the presence or absence of TNFα (200 U/ml) and oATP 500 microM. Freshly obtained PMBC (peripheral blood mononuclear cells) by means of fycoll from buffy-coat were added in the upper compartment and allowed to migrate over night to the lower chamber which contained RPMI and 10% fetal calf serum. At the end of the culture, migrated cells were recovered from the lower chamber and counted FIG. 4 shows the transendothelial migration assay. The measure of migrated PBMC through a HUVEC monolayer indicates that the addition of TNFα (200 U/ml) significantly increases the number of migrated PBMC, expressed as percentage over basal, whereas the addition of 500 microM oATP significantly reduces the TNFα-induced transendothelial migration. Data represent mean±SEM of 7 experiments.

In another series of experiments, we tested the possible direct effect of oATP on tumor cell growth. We used, both in vitro and in vivo, the RMA cells.

In Vitro Experiments

RMA cells were derived from the Rausher leukemia virus-induced mouse T-cell lymphoma RBL-5 of B6 origin.

Cell Isolation and Cultures

RMA wild type (wt) murine lymphoma T cell line (ATCC) was grown in RPMI 1640 supplemented with 10% heat inactivated fetal bovine serum, 100 U/ml penicillin, 100 microg/ml streptomycin.

Lymphocytes were obtained from a buffy-coat by a fycoll gradient and depleted from monocytes by adherence.

Lymphocytes were activated with Phytohaemoagglutinin A mitogen (PHA).

Cell Apoptosis Assay

Apoptosis was evaluated by FACS analysis, after staining with annexin-V FITC-conjugate to show the exposure of phosphatidyl serine on the external side of plasma membranes, and with propidium iodide (PI).

Two thousand RMA wt cells and lymphocytes were treated with or without different concentrations of oATP for 24 and 48 hours. The cells were then washed with PBS with Ca++ and Mg++ and stained.

Ten thousand cell/sample were analysed and the percentage of annexin V/PI positive cells was calculated with FCS-express software (DE NOVO Software).

Cell Cycle Analysis

RMA wt cells were treated with different concentrations of oATP for 24 and 48 hours, then fixed for at least four hours in ice-cold 70% ethanol. Cells were stained with a solution containing NP-40, RNase and PI and cell cycle distribution was detected using FACScan (Becton Dickinson, San Jose, Calif.) and analyzed with FCS-express software.

The obtained results indicate that oATP, at high concentrations, is able to induce apoptosis of RMA cells and not of lymphocytes.

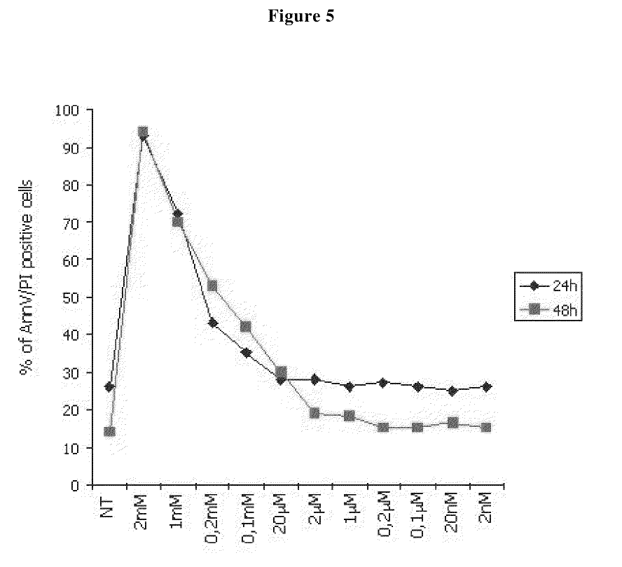

Sensitivity of RMA wild type cell line to different concentrations of oATP evaluated as percentage of AnnexinV-PI positive cells at 24 and 48 hours of treatment (FIG. 5).

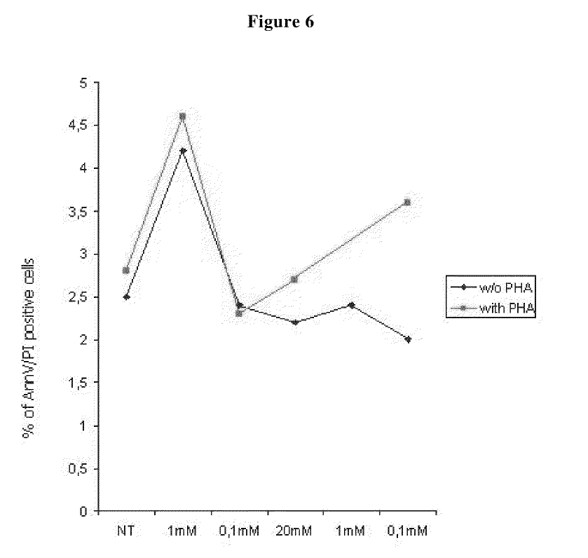

Sensitivity of lymphocytes from buffy-coat (PMBC), activated or not with PHA, at different concentrations of oATP, evaluated as percentage of AnnexinV-PI positive cells at 24 hours of treatment (FIG. 6).

The values of the cell cycle progression are reported in the Table.

TABLE

| | | | | % G2/ |
|---|---|---|---|---|
| | % subG1 | % G0/G1 | % S | M |
| oATP24 h | | | | |
| NT | 8 | 42 | 32 | 18 |
| 1 mM | 75 | 16 | 6 | 3 |
| 0.1 mM | 31 | 44 | 20 | 5 |
| 1 μM | 23 | 38 | 29 | 10 |
| 0.1 μM | 9 | 48 | 24 | 19 |
| oATP48 h | | | | |
| NT | 10 | 50 | 24 | 16 |
| 1 mM | 24 | 44 | 25 | 7 |
| 0.1 mM | 14 | 33 | 33 | 20 |
| 20 μM | 8 | 50 | 23 | 19 |
| 1 μM | 3 | 52 | 19 | 26 |
| 0.1 μM | 4 | 50 | 24 | 22 |

Cell cycle progression of RMA wt cell line treated with different concentrations of oATP for 24 or 48 hours.

Comment

At high doses of oATP, there is a high mortality of RMA wt cells by apoptotic mechanism, as demonstrated by sub G1 peak, in accordance with the percentage of AnnexinV/PI positive cells (FIG. 5). Concomitantly, surviving cells loss partially the capacity to entry in S and/or G2/M Phases of the cell cycle, mainly at 24 hours of treatment.

When the concentration of the oATP decreases, cells display a trend similar with the control one.

At high dose of oATP (1 mM) and 48 hours of treatment, there is a paradoxical decrease in the percentage of cell death, assessed by sub G1 peak, at variance with the percentage of AnnexinV/PI positive cells which strongly increases (FIG. 5). This could depend on the proliferation of cells escaped from the treatment.

To note that, at low doses, there is increase of the percentage of cells in the G2/M phase of the cell cycle.

### Example 4

RMA cells were derived from the Rausher leukemia virus-induced mouse T-cell lymphoma RBL-5 of B6 origin and maintained in RPMI 1640 medium, supplemented with fetal bovine serum, 1% penicillin/streptomycin and 1% glutamine (complete medium).

C57BL/6 female mice weighing about 18-20 g (8 week old) were used. The cells were washed twice with 0.9% NaCl and subcutaneously injected in each mouse in a volume of 100 microliters containing 7×10⁴ cells. After 10 days from RMA cell injection, we treated the mice with oATP. Five groups of mice were studied (each of 7 mice): 1) controls, untreated; 2) locally (subcutaneously) (sc) treated with 0.5 mg of oATP; 3) locally (sc) treated with 1 mg oATP; 4) intraperitoneally treated with 1 mg oATP; 5) locally (sc) treated with 1 mg oATP for 3 days only.

The mice were observed until the 20^{th} day from the tumor inoculation: they were weighed daily and the size of the tumor mass was daily measured.

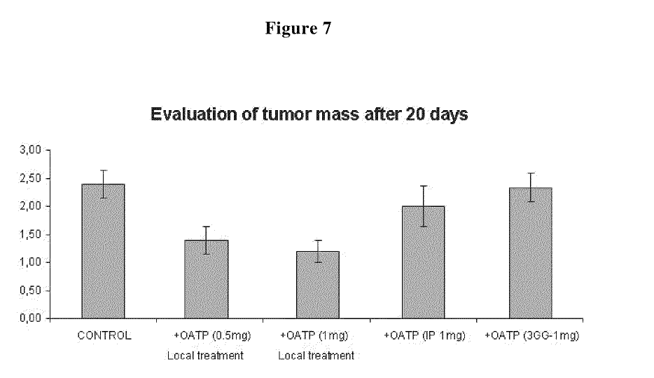

The tumor mass (measured by arbitrary units) did not significantly grow in the groups 3 and 5 for three days from the beginning of the treatment (e.g. until the 14^{th} day). Successively, the control of the tumor growing is mainly exerted until the number of the tumor cells is not exponentially expanded. However, at 20 days from RMA cell injection, in the group 3 the tumor growth was less evident than that observed in the other groups (FIG. 7); in addition, the tumor mass obtained in 20 days in the mice from groups 3 was less solid than the tumor mass obtained in the other groups.

No significant differences were observed between the body weights of the mice measured during the different treatments.

Our data show that the local continuous treatment with 1 mg oATP is efficient in significantly slowing the tumor growth. It is possible that more elevated doses of oATP necessitate to control the tumor growth in the time.

Our in vitro data suggest that elevated concentrations of oATP (1 mg for about 70,000 RMA cells) are able to induce the apoptosis of these cells.

### Example 5

We compared the effects of oATP to that of two known compounds able to antagonize the P2X7 receptors: 1) the pyridoxal phosphate-6-azophenil-2′, 4′-disulphonic acid (PPADS), a non-specific P2X7 antagonist, and 2) 1-(N,O-bis[5-isoquinolinesulphonyl]-N-methyl-L-tyrosyl)-4-phenylpiperazine (KN62), a potent P2X7 antagonist (especially at the human receptor).

We used ATP (able to increase the P2X7 receptor expression on the cells), and the P2X7 inhibitors, oATP, PPADS and KN62 at the concentrations reported in the literature, e.g.: ATP 3 mM, oATP 300 microM, PPADS 50 microM and KN62 10 microM.

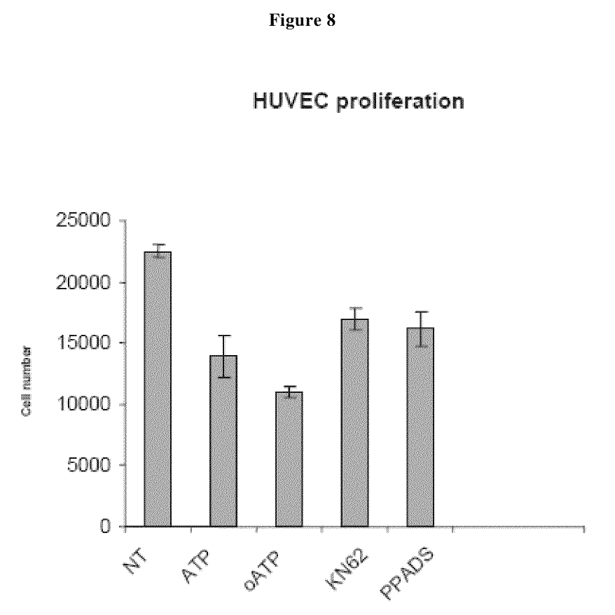

In the first series of experiments we studied the proliferation of human umbilical vein endothelial cells (HUVEC), obtained as previously described (1). The HUVEC were treated with each of the compounds for 36 hours, washed and fixed with glutaraldeide 2% in PBS. The cells were colored with crystal violet 0.1%, washed and dried. The dye solubilization was performed with acetic acid 10% and the absorbance was measured spectrophotometrically at 595 nm, using a microplate reader. The optical density was proportional to the number of cells. As reported in the FIG. 8, cell growing number is significantly reduced by all the treatments (mean±SEM of 7 experiments), but the maximal reduction of the proliferation has been induced by oATP.

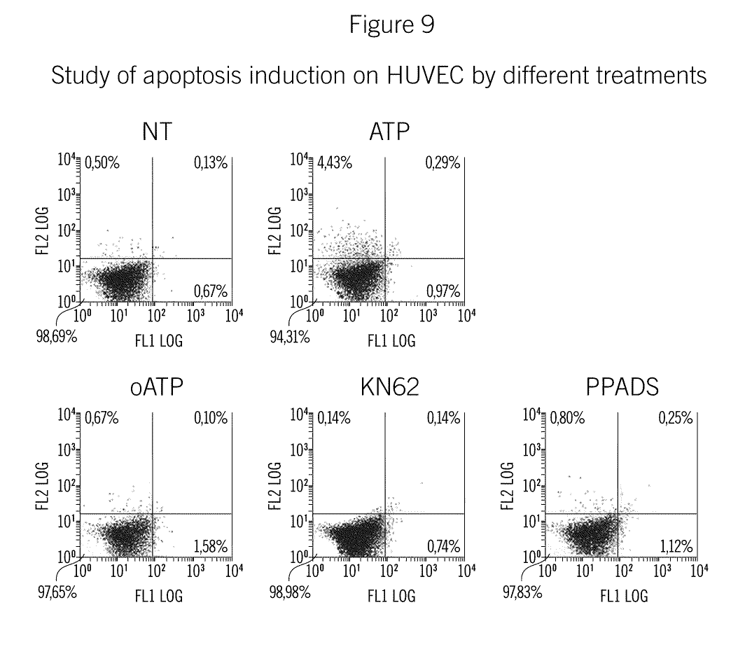

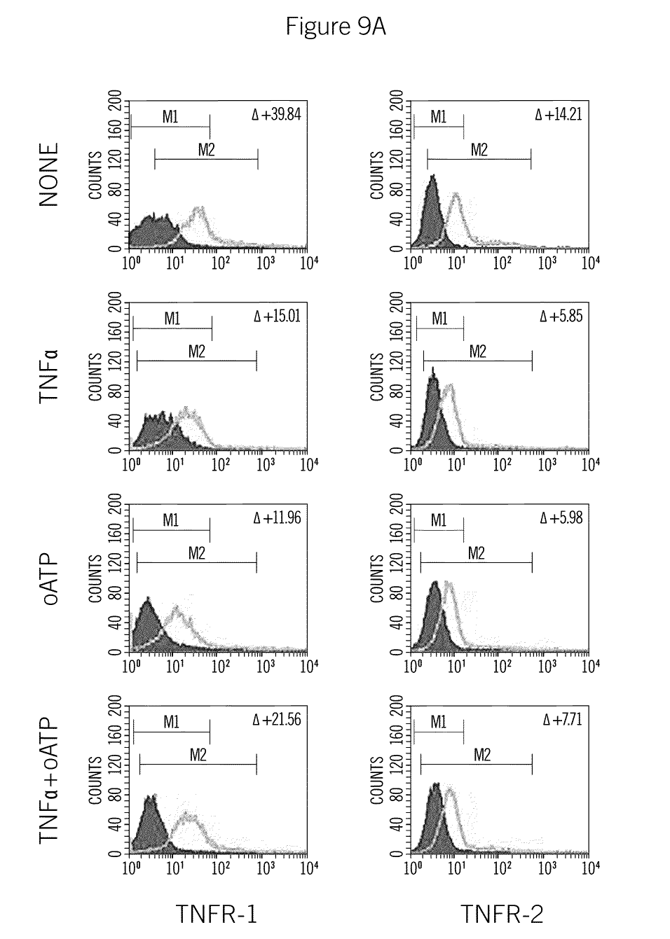

It is possible that the inhibition of the ATP-dependent pathway can induce the shift to another metabolic activity, as the glycolysis. We studied if the compounds were able to induce apoptosis or necrosis of the HUVEC, by staining the cells with annexin V and propidium iodide (see the following description). The block of the HUVEC proliferation is further evidenced by the absence of cell apoptosis due to the treatment of the cells with the tested compounds, as reported in the FIG. 9 (the results are representative of 5 experiments). Only the treatment with ATP was able to induce about 4.5% PI positive, e.g. necrotic cells.

In addition, oATP was cytofluorometrically assayed for its ability to modulate the expression of TNFalpha receptors on endothelial cells. As reported in the FIG. 9a, the use of human TNFalpha (100 U/ml) improved the expression of the TNFalpha receptor 1 (TNFR1) on HUVEC. The addition of oATP alone did not modify such expression. However, oATP was able to downregulate the TNFalpha-induced increased expression of TNFR1.

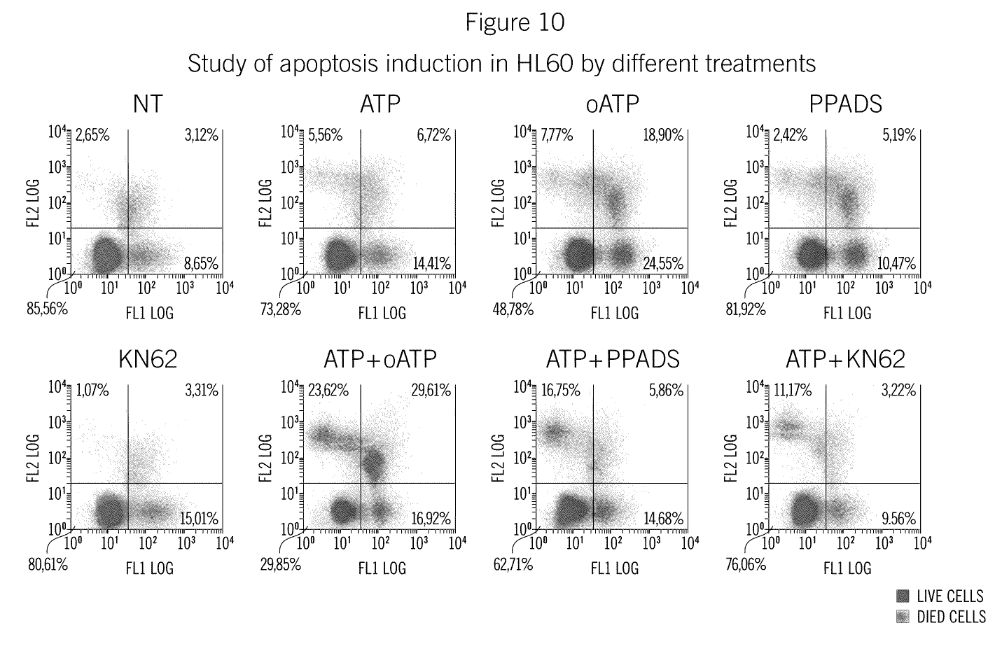

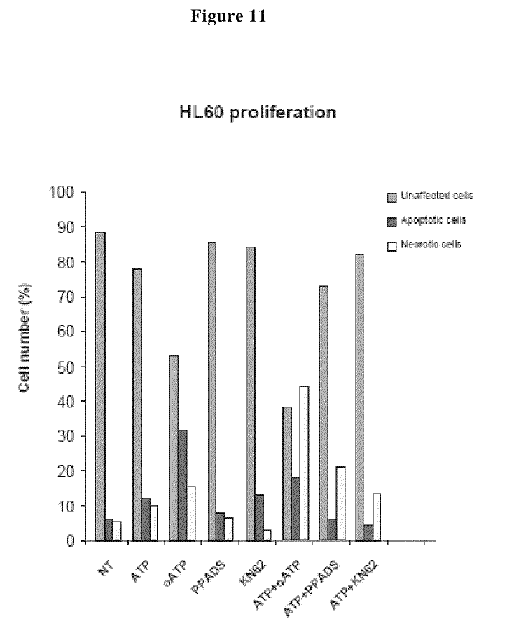

In the second series of experiments, we studied the differences in the apoptosis induction on human promyelocytic leukemia HL60 cells by ATP and by the inhibitors of the P2X7 receptors. Apoptotic cells and necrotic cells were analyzed by staining the cells with annexin V and propidium iodide (PI) (BD Pharmingen apoptosis kit, San Diego, Calif.). Briefly, an aliquot of 10⁵ cells was incubated with annexin V-fluorescein isothiocyanate (FITC) and PI for 15 minutes at room temperature in the dark. The cells were immediately analyzed by FACScalibur (Becton Dickinson, Heidelberg, Germany). The emission/excitation wavelengths were 530/488 nm for annexin V FITC (FL1) and 640 nm/488 nm for PI (FL2). The necrotic cells were annexin V- and PI-positive, whereas apoptotic cells were annexin V-positive and PI-negative. The percentage of cells stained in each quadrant was quantified using the cellQuest software (BD Bioscience, San Josew, Calif.). The data, reported in the FIG. 10, and representative of 5 experiments, show that oATP was able to induce cell apoptosis at better extent with respect to PPADS and KN62 and such effect was dependent on the concentration of oATP. The combined use of ATP with one of the P2X7 inhibitors increased the number of necrotic cells. FIG. 11 summarizes the effects of the different treatments on the apoptosis/necrosis induction in the HL60 cells.

What is claimed is:

1. A method for treating a tumor disease selected from lymphoma and leukemia in a subject in need thereof, said method comprising administering to said subject an effective amount of adenosine-5′-triphosphate-2′,3′-dialdehyde (oATP).

2. A method for inhibiting vascular endothelial growth factor (VEGF)-induced cell proliferation of endothelial cells, comprising: administering to said endothelial cells an effective amount of adenosine-5′-triphosphate-2′,3′-dialdehyde (oATP).

3. The method according to claim 2, wherein the endothelial cells are human endothelial cells.

4. The method according to claim 1, wherein the adenosine-5′-triphosphate-2′,3′-dialdehyde is administered in a pharmaceutical form selected from the group consisting of tablet, pill, capsule, granulate, powder, suppository, solution, spray, syrup, cream, ointment, gel, paste, lotion, emulsion and suspension.

5. The method according to claim 1, wherein the adenosine-5′-triphosphate-2′,3′-dialdehyde is administered in a daily dosage of 0.01 to 100 mg per Kg of body weight of said subject.
